(19) United States
(12) Patent Application Publication
(10) Pub. No.: US 2021/0169666 A1
(43) Pub. Date: Jun. 10, 2021

(54) SYSTEM FOR INTRODUCING A BREAST IMPLANT

(71) Applicant: R. Alastair Winn, Santa Barbara, CA (US)

(72) Inventor: R. Alastair Winn, Santa Barbara, CA (US)

(21) Appl. No.: 16/708,606

(22) Filed: Dec. 10, 2019

(51) Int. Cl. A61F 2/52; A61B 17/00; A61F 2/00

(52) U.S. Cl. CPC A61F 2/52; A61B 17/00; A61B 2017/00544; A61B 2017/00796; A61F 2/0059

(57) ABSTRACT

A system and device for providing enhanced delivery of an implant into a surgical pocket is described.

### BACKGROUND

The present invention is generally directed to instruments and devices for introducing an implant into a patient through a relative small incision. More specifically, the invention is directed to a system utilizing pressurized air or other gas against an extrusion force applicator to force the implant through an opening of an nozzle and into a surgical pocket in the patient.

Implantable breast prostheses have been in worldwide use for a number of years. One problem with the insertion of implantable breast prostheses is that the implantable prostheses are often provided in a filled condition and must be removed from the package and manually inserted into a surgical pocket. As a result, traditional surgical approaches have required the use of relatively large incisions. Furthermore, manual insertion can lead to damage of the fragile implant due to high localized stresses applied during insertion.

Recently, a number of insertion aides have been developed to reduce the size of the incision needed and to ease the actual implantation of the implantable breast prosthesis. One such aide is a flexible funnel shaped device having a large proximal opening into which an implantable breast prosthesis may be placed, and a smaller distal opening which may be placed into an appropriately sized incision in the patient. To work efficiently, existing insertion aides require a lubricant or prewetting to activate the lubricious low friction surface. Furthermore, the implant must be removed from the sterile package, thus potentially introducing biological contamination. The implantable breast prosthesis is then inserted through the incision by manually squeezing the funnel shaped device to forcibly express the implantable breast prosthesis through the distal opening past the incision site and into the surgical pocket.

It has been observed that some versions of a flexible funnel shaped device to implant the breast implant may be difficult in view of the relatively large compression force that must be imparted to the funnel by a surgeon's hands to express the implant through the distal opening of the funnel.

What has been needed, and not previously available, is a system that allows the implant and introducer to be lubricated without compromising the sterility of the implant and introducer, and also provides for hydraulic or pneumatic expression of the implantable breast prosthesis through the distal opening incorporated into the sterile package into a patient's body in a controllable manner. The present invention satisfies these and other needs.

### SUMMARY OF THE INVENTION

In its most general aspect, the present disclosure describes a system using a hydraulic or pneumatic force driven actuator or bladder to extrude a pre-filled breast implant out of a closed container through a small nozzle, thus allowing touchless aseptic insertion into a surgical pocket.

In another aspect, the present disclosure describes a device for use as an introducer for assisting in implanting an implantable device in a body pocket, comprising: an introducer body having a holding portion disposed at distal end of the introducer and a nozzle portion disposed at a proximal end of the introducer body; an extrusion force applicator mounted over a distal opening of the introducer; a cap mounted over the extrusion force applicator and configured to be attached to the introducer body; a port mounted on a distal end of the cap, the port configured to receive a source of gas or fluid; and an injection port disposed at a proximal end of the nozzle port, the injection port configured to provide resealable access to an interior of the introducer body.

In one alternative aspect, the extrusion force applicator is a diaphragm. In another alternative aspect, the extrusion force applicator is a bellows. In another alternative aspect, the extrusion force applicator is an expandable balloon. In yet another alternative aspect, the implantable device is a breast implant.

In yet another aspect, the present disclosure further discloses a source of gas or fluid, the source of gas or fluid in fluid communication with the port.

In still another aspect, the present disclosure describes a method for implanting an implant into a body pocket, comprising: applying pneumatic or fluid pressure to an extrusion force applicator configured to apply an extrusion force against an implant in response to the pneumatic or fluid pressure to force the implant through a nozzle of an introducer into a body pocket.

In still another aspect, the disclosure further describes adding lubricant to an interior of the introducer to lubricate the interior of the introducer, extrusion force applicator, and the implant. In one aspect, the lubricant is bioresporbable. In another aspect, the introducer is a volume restricted container, the volume restricted container is capable of being sterilized.

In another aspect, the extrusion force application is selected from the group consisting of a diaphragm, an expandable balloon, and a bellows.

In a further aspect, the method includes connecting a pressure source to a port of the introducer to apply pressure to the extrusion force applicator to extrude the implant from the introducer. In one aspect the pressure source is compressed air. In another aspect, the pressure source is hydraulic in nature.

In still another aspect, the lubricant coats the interior of the introducer, the extrusion force applicator, and the implant to reduce friction between the implant, introducer, and extrusion force applicator.

In yet another aspect, the disclosure describes a sterilizable volume restricted container for assisting in implanting an implantable device in a body pocket, comprising: a volume restricted container having a holding portion disposed at distal end of the introducer and a nozzle portion disposed at a proximal end of the introducer body; an expandable volume reducer mounted over a distal opening of the introducer; a cap mounted over the expandable volume reducer and configured to be attached to distal end of the volume restricted container; a port mounted on a distal end of the cap, the port configured to receive a source of gas or fluid; an injection port disposed at a proximal end of the nozzle port, the injection port configured to provide access to an interior of the introducer body.

In another aspect, the sterilizable volume restricted container is reusable. In yet another aspect, the cap is formed of aluminum, the aluminum cap providing increased thermal conduction to contents of the sterilizable volume restricted container and an interior of the sterilizable volume restricted container to reduce dry heat sterilization cycle time.

In still another aspect, the expandable volume reducer is a diaphragm. In yet another aspect, the expandable volume reducer is a balloon. In still another aspect, the expandable volume reducer is a bellows.

In yet another aspect, the implant contained in the volume restricted container is made with high strength gel with a compressive rupture force greater than 15 kg using a 15 mm diameter probe. In another aspect, the expandable volume reducer is configured to maximize extrusion force and minimize radial compression forces.

In still another aspect, the expandable volume reducer is a ribbed diaphragm with variations in thickness to direct forces during compression. In another aspect the expandable volume reducer is a bellows with variations in thickness to direct forces during compression. In still another aspect, the expandable volume reducer is a multi-balloon expander configured to direct forces during compression. In yet another aspect, the expandable volume reducer is a diaphragm wherein the center of the diaphragm is tethered to maximize compressive forces at the edges of the volume restricted container and minimize radial forces during implantation of an implant through the nozzle portion.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a side perspective view of a prior art funnel-shaped introducer used to facilitate implantation of an implant through a narrow opening of the introducer into a body.

FIG. 2 is a cutaway view of the introducer of FIG. 1 also showing a breast implant positioned within the introducer, with a nozzle portion of the introducer extending through an surgically created opening in the body of a patient.

FIG. 3 is a side cross-sectional view illustrating an embodiment of the disclosure illustrating the placement of an introducer containing an implant within a sterile package.

FIG. 4 is an exploded cross-sectional view showing details of the introducer and implant depicted in FIG. 3.

FIG. 5 is a cross-sectional view of embodiment of FIG. 4 depicting a sterile introducer and implant ready for implantation, with a source of air or fluid fluidly connected to the introducer for use to pneumatically or hydraulically express the implant through a nozzle portion of the introducer; the position and shape of the extrusion force applicator and the position of the implant is shown before pneumatic or hydraulic pressure is applied to the introducer.

FIG. 6 is a cross-sectional view of embodiment of FIG. 5 showing the shape and position of the extrusion force applicator and the position of the implant after pneumatic or hydraulic pressure is applied to the introducer.

### DETAILED DESCRIPTION OF THE PREFERRED EMBODIMENTS

As will be described hereinafter in greater detail, the various embodiments of the present invention relate to an apparatus and method for facilitating insertion of an implantable breast prosthesis. For purposes of explanation, specific nomenclature is set forth to provide a thorough understanding of the present invention. Description of specific applications and methods are provided only as examples. Various modifications to the embodiments will be readily apparent to those skilled in the art and the general principles defined herein may be applied to other embodiments and applications without departing from the spirit and scope of the invention. Thus the present invention is not intended to be limited to the embodiments shown, but is to be accorded the widest scope consistent with the principles and steps disclosed herein.

In describing the various figures herein, the same reference numbers are used throughout to describe the same element that appears in more than one embodiment of the present invention. Detailed descriptions of various elements that appear in more than one embodiment is not repeated in the descriptions of following figures, even though such element is labeled with the same reference number.

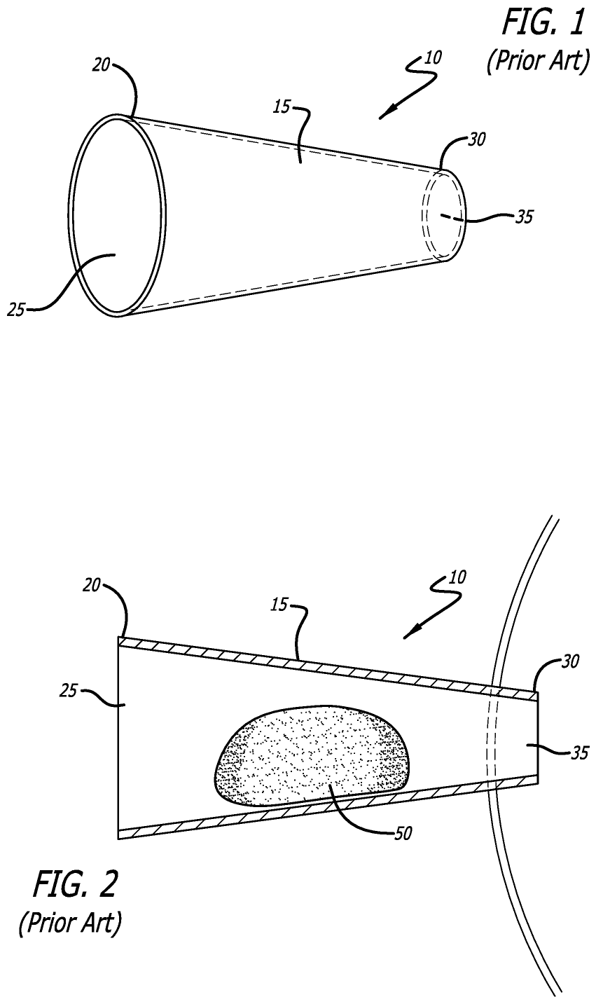

FIG. 1 illustrates one embodiment of a prior art breast implantation funnel 10 in accordance with various principles of the present invention. The funnel 10 has a flexible body 15 having a proximal end 20. Proximal end 20 has an opening 25 that is sized to receive a breast prosthesis to be implanted into a patient. Funnel 10 also has a distal end 30 having an opening 35.

Typically, the flexible body 15 of the funnel 10 is comprised of a flexible and transparent material. Such materials may include medical grade flexible plastic materials such as PVC mixed with a suitable plasticizer, ethylene vinyl acetate or a polyolefin, such as polypropylene, or a medical grade silicone elastomer. Such materials are flexible, strong and are capable of slightly stretching without rupture. It is also useful if the material used to form the body of the funnel is transparent or translucent so that the orientation of the breast prosthesis may be directly observed as it is being implanted.

FIG. 2 illustrates the use of the funnel 10 to assist the implantation of a breast prosthesis 50. As shown, breast prosthesis 50 is inserted into the opening 25 disposed at the proximal end 20 of the funnel. The prosthesis 50 is typically manipulated through the body 15 of the funnel towards the opening 35 disposed in the distal end 30 of the funnel. Because of the slope of the funnel which results in the diameter of opening 35 being smaller than that of opening 25, there is a point where the prosthesis becomes lodged within the funnel. At that point, the surgeon may take hold of the funnel in the proximity of the proximal end 20 and begin the squeeze the outer wall of the body 15 to impart pressure on the prosthesis in a distal direction, ultimately causing the prosthesis to be expelled through opening 35 in the distal end 30 of the funnel into a cavity formed within the patient's chest.

In some prior art uses, a lubricating substance may be coated on an inner wall of the funnel body 15. The coating may, for example, consist of an ionic lubricant, including hydrophilic lubricants as manufactured by, or similar to, those manufactured by Advanced Biomaterials, AST Products, Biocoat, Coatingstogo, DSM, Harland Medical Systems, Surface Solutions Group or PolyBioMed. Other conventional commercially available surgical lubricants can also be used, such as Surgilube™, or gels, such as, for example, Aquasonic™ and the like that may or may not contain common antibiotics such as bacitracin or other antimicrobial (antibacterial, antiviral, antifungal) agents.

In further prior art uses, the surface of the inner wall of the funnel body may be made more lubricious by forming a hydrophilic coating on the surface of the inner wall. As is known in the art, surfaces have been rendered hydrophilic by such methods as high energy radiation in situ polymerization processes, by direct chemical bonding or by forming interpolymer networks. The radiation process can render a very stable hydrophilic surface, but suffers from unreliable results and can produce radiation damage to the substrate. Formation of interpolymer networks also produces hydrophilic surfaces but in turbulent flow or extended soaking, the interpolymer networks often break down and the hydrophilic portion can be washed away rendering the substrate surface defective.

Other methods described in the art use a polyurethane coating agent to adhere poly-N-vinyl pyrollidone (PVP) to various substrates, thus producing an article having a hydrophilic coating of low coefficient friction. Extensive studies indicate, however, that in turbulent flow or upon extended soaking in aqueous media, the hydrophilic coating can be leeched off, thus rendering the article insufficiently hydrophilic. Another method for creating a hydrophilic coating on an article, such as, for example, the funnel-shaped body described above involves coating the inner wall of the funnel-shaped body with a polyisocyanate and a hydrophilic copolymer having pendant groups which react with the isocyanates. The polyisocyanate and the hydrophilic copolymer produce a covalent graft or bond between the hydrophilic coupling agent and the hydrophilic copolymer.

In one such prior method, the inner wall of the funnel-shaped body may be is exposed to a polyisocyanate in a solvent solution by dripping, spraying or the like and then evaporating the solvent preferably by air drying. This step forms a coating with unreacted isocyanate groups on the surface of the substrate. A copolymer having an average of at least two active hydrogen atom sites per molecule is then applied to the surface of the substrate and reacts with the unreacted isocyanate groups to produce a covalently bound matrix, thus forming a stable hydrophilic coating.

A more detailed explanation of exemplary processes and materials that may be used to form a bioresporable lubricious layer of low friction hydrophilic matter on the inner wall of the funnel-shaped body is described by Winn in U.S. Pat. No. 4,373,009, the entirety of which is hereby incorporated herein.

Due to typical high friction between the funnel-shaped device and a silicone implant, additional lubrication is usually necessary. Surgical lubricants have been used to utilize a hydrophilic coating on the insertion sleeve, and a mechanical device has been used wherein the funnel-shaped device is placed in a cylinder with a rigid piston that forces the implant through a nozzle. All of the methods utilized in the prior art require the sterile implant to be removed from its packaging is a dry and unlubricated state, and inserted into a secondary device which may or may not have lubricant to aid in the passage of the implant through the funnel.

Typically, the internal surface of the funnel-shaped device is treated with the lubricant, and the internal surface requires pre-wetting of the funnel to activate the dry hydrophilic coating to render the coating lubricious. During this re-wetting procedure, sterility is compromised.

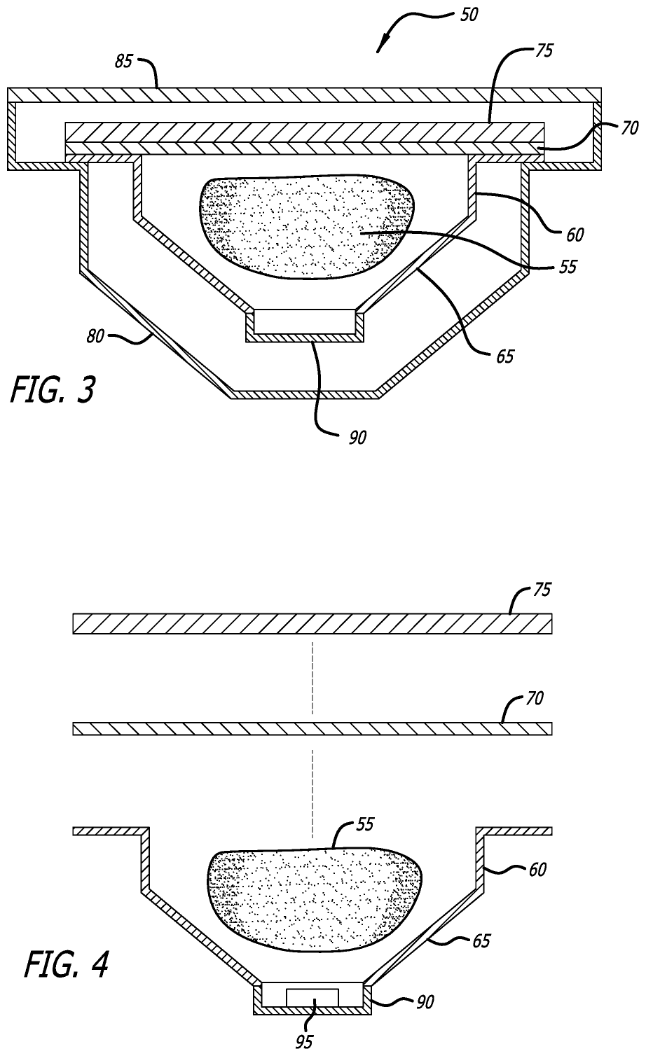

FIG. 3 depicts one embodiment 50 of an introducer of the present disclosure shown enclosed in a sterile package. An implant 55 is enclosed within an introducer 60 having a nozzle portion 65. An elastic extrusion force applicator 70 is shown disposed over a proximal end of the introducer 60. A cap 75 is mounted to the introducer distally to the extrusion force applicator, and configured to lock onto the introducer 60. The entire introducer assembly and implant is enclosed in a sterile package having a proximal portion 80 sized to accept the introducer assembly. The sterile package is sealed by a top portion 85 in such a way that the contents of the package are maintained in a sterilized state once sterilization of the package and introducer assembly has been accomplished.

FIG. 4 is an exploded cross-sectional view of the implant and introducer assembly. In this figure, implant 50 is positioned within introducer 60. Extrusion force applicator 70 is disposed over the distal top of the introducer. Cap 75 is then used to seal the extrusion force applicator to the introducer. Also shown is a nozzle 65 disposed at a proximal end of the introducer, through which the implant will be expressed into an opening in a patient's body. In some embodiments, the nozzle may be closed with a cap 90, in or over which a thin membrane 95 is disposed to maintain the contents of the introducer within the introducer.

The thin membrane may also be configured to form a needle port through which a material, such as, for example, a sterile lubricant may be aseptically injected into the interior of the introducer. Once the lubricant or other material is inserted into the interior of the introducer, the lubricant may be distributed throughout the interior of the introducer and the surface of the implant by shaking the introducer. In this manner, any friction between the surface of the implant and the surface of the nozzle portion of the introducer may be reduced to ease passage of the implant through the nozzle portion and into an opening of the body of a patient.

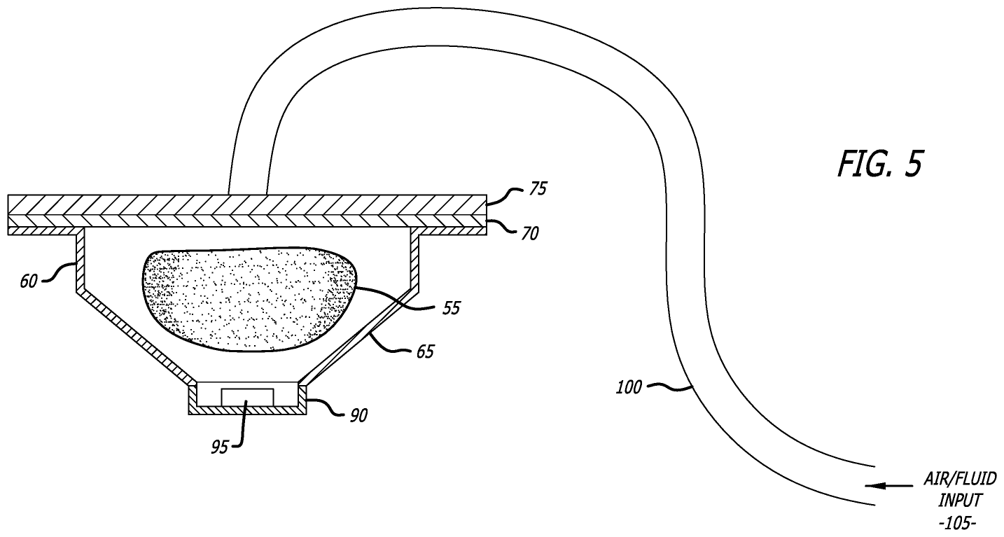

FIG. 5 depicts an embodiment showing introducer 60 being connected to a hose that is in turn connected to a controllable source of gas or fluid 105. When the gas or fluid control is activated, gas or fluid flows through hose 100 into a space located between the cap 75 and the distal surface of extrusion force applicator 70. As gas or fluid is controllable transmitted to the introducer, the extrusion force applicator stretches toward the nozzle of the introducer as the pressure in the introducer increases. When the pressure reaches a sufficient level, the extrusion force applicator pushes the implant 55 towards nozzle portion 65, positioning the implant for expression through the nozzle and into an opening in a patient's body.

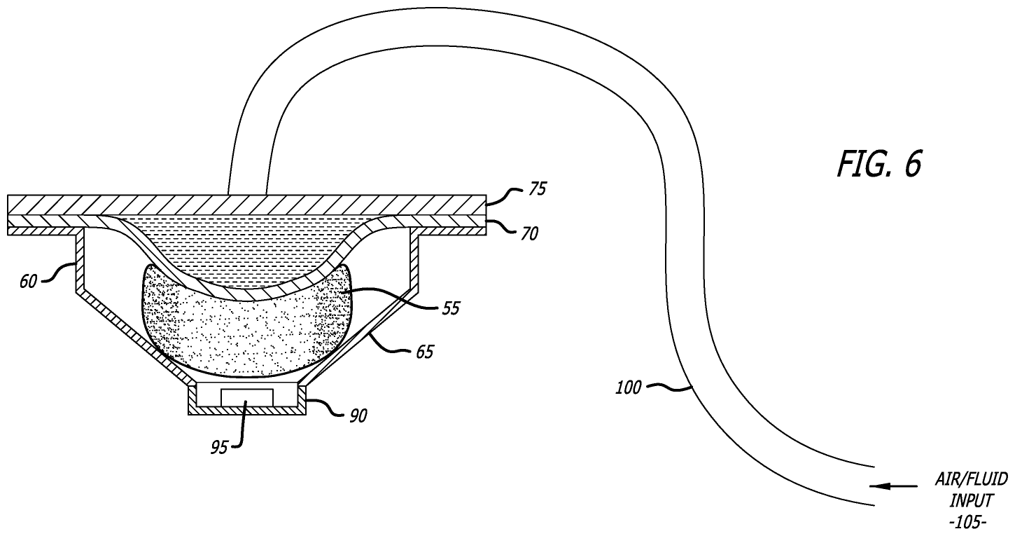

FIG. 6 illustrates how the extrusion force applicator stretches and pushes the implant towards and through the nozzle 65 as the pressure applied to the distal surface of the extrusion force applicator is increased. As is easily understood, the implant will eventually be expressed through the nozzle and into the surgical pocket.

The extrusion force applicator may be an elongatable diaphragm, bellows or balloon, made from a suitable extensible and biocompatible material. The inventor has observed that extrusion of an implant can be accomplished with pressures in the range of 2-20 pounds per square inch, and preferable around 5 pounds per square inch. The thickness of the extrusion force applicator may be selected to be sufficiently rigid to retain shape and withstand mild pressure during the application process. While the applicator package/assembly can be flexible, it is not necessary. Additionally, various safety methods may be employed that the pressure being supplied to the extrusion force applicator does not exceed a pre-determined strength of the extrusion force applicator.

The introducer may be manufactured from a biocompatible rigid material that may be molded or cast using well known technologies. For example, one material which can be used is polycarbonate, because of its high temperature resistance and optical clarity, thus enabling dry heat sterilization and visualization during the insertion process.

The design of the introducer nozzle is important. It is desirable that the package be minimized in height for package design and storage purposes. It is also desirable to minimize opposing vector forces resisting the force propelling the implant through the nozzle. A simple X-Y vector analysis can enlighten the designer. In a 1:1 aspect ratio of height to width, the slope is 45 degrees. Thus 50% of the downward force caused by the extension of the extrusion force applicator is transferred to a lateral force. A typical silicone breast implant is a thin silicone elastomer bag filled with a soft malleable cohesive gel as described in U.S. Pat. No. 3,293,663. This silicone gel inside the implant is fragile and can be irreversible crushed by compressive forces, as would be applied by the extrusion force applicator herein described. For example, a 15 mm diameter cylindrical probe travelling at 25 mm/min is used to test “crush strength”. Older gel formulations such as Dow Corning Q7-2167/2168 can be crushed with between 10 and 4 and 8 kg force. New high strength gels, such as Applied Silicone Corporation 400135 can achieve crush strengths of over 20 kg. The strength of the gel is important because the force during extrusion through a small orifice can cause irreversible crushing and deformation of the implant. While lubrication minimizes those resistive vector force transfers, it is not possible to eliminate all of them. Thus, it is desirable to combine a high strength gel filled breast implant in a package with minimal vector forces transferred to the implant along the axis of extrusion.

The design of the extrusion force applicator may further serve to minimize undesirable crushing forces. For example, a extrusion force applicator design that maximizes extrusion force along the perimeter of the package and minimizes compressive radial forces at the center is most desirable. This can be done by variations in thickness of the extrusion force applicator or addition of bulk modulus increasing “ribs” on the extrusion force applicator. It can also be accomplished with a bellows designed to maximize compelling force and minimize compressive radial forces. In the case of a balloon type bladder, the thickness of the balloon wall can be varied to increase force in one direction. Also, a bladder consisting of a combination of diaphragm, bellows and multiple inflatable balloons can be used. Alternatively, a flexible tether can be used to restrict elongation in the center of a balloon type bladder, while maximizing elongation and force at the outside edges of the bladder. Those skilled in the art will understand that there are many possible variations of the design and configuration of the disclosed inventions so as to minimize radial force and maximize extrusion force applied to the implant as it travels through the nozzle.

The hydraulic or pneumatically driven insertion device must be of sufficient strength to withstand and direct the extrusion force toward the nozzle. There are many ways to design such a device or package, and such a device or package will all these following common characteristics: the air or hydraulic fluid will not directly contact the implant; the package or device will be of sufficient rigidity or strength and integrity to withstand the radial and directed forces of the extrusion force applicator; the package or device will have a provision to add lubricant prior to extrusion of the implant to allow the nozzle, implant and extrusion force applicator to be coated to reduce friction and prevent damage of the implant during insertion. Additionally, the package or insertion device should be sterilizable, typically by dry heat to 130 degrees Centigrade, but autoclaving or ethylene oxide (ETO) may also be used.

Any biocompatible gas or fluid may be used to apply pressure to the extrusion force applicator to extrude the implant from the nozzle of the introducer. Moreover, various devices used to pressurize the biocompatible gas or fluid may be used to force the gas or fluid against the extrusion force applicator to force the extrusion force applicator to extrude the implant from the nozzle of the introducer. For example, a hand pump attached to hose connected to the introducer may be used to apply pressure to the extrusion force applicator, and thus the implant. Alternative, a mechanical pump, electrically operated pump, gas cylinder, gas compressor, pressurized fluid container, and the like may be used.

While the use of a lubricant to reduce friction between the introducer, the extrusion force applicator, and a breast implant is advantageous, such use is not necessary to accomplish the benefits of the system and device described in this disclosure. Any lubricant that may be used should be biocompatible and preferably bioresporbable. Suitable lubricants are described by Winn in U.S. Pat. No. 4,731,081, the entirety of which is hereby incorporated herein. The lubricant can be incorporated into the sterile package by the manufacturer or injected into the package shortly before surgical insertion.

While particular embodiments of the present invention have been described, it is understood that various different modifications within the scope and spirit of the invention are possible. The invention is limited only by the scope of the appended claims.

I claim:

1. A device for use as an introducer for assisting in implanting an implantable device in a body pocket, comprising:
an introducer body having a holding portion disposed at distal end of the introducer and a nozzle portion disposed at a proximal end of the introducer body;
an extrusion force applicator mounted over a distal opening of the introducer;
a cap mounted over the extrusion force applicator and configured to be attached to the introducer body;
a port mounted on a distal end of the cap, the port configured to receive a source of gas or fluid; and
an injection port disposed at a proximal end of the nozzle port, the injection port configured to provide resealable access to an interior of the introducer body.

2. The device of claim 1, wherein the extrusion force applicator is a diaphragm.

3. The device of claim 1, wherein the extrusion force applicator is a bellows.

4. The device of claim 1, wherein the extrusion force applicator is an expandable balloon.

5. The device of claim 1, wherein the implantable device is a breast implant.

6. The device of claim 1, further comprising a source of gas or fluid, the source of gas or fluid in fluid communication with the port.

7. A method for implanting an implant into a body pocket, comprising:
applying pneumatic or fluid pressure to an extrusion force applicator configured to apply an extrusion force against an implant in response to the pneumatic or fluid pressure to force the implant through a nozzle of an introducer into a body pocket.

8. The method of claim 7, further comprising adding lubricant to an interior of the introducer to lubricate the interior of the introducer, extrusion force applicator, and the implant.

9. The method of claim 7, wherein the lubricant is bioresporbable.

10. The method of claim 7, wherein the introducer is a volume restricted container, the volume restricted container is capable of being sterilized.

11. The method of claim 7, wherein the extrusion force application is selected from the group consisting of a diaphragm, an expandable balloon, and a bellows.

12. The method of claim 7, further comprising connecting a pressure source to a port of the introducer to apply pressure to the extrusion force applicator to extrude the implant from the introducer.

13. The method of claim 12, wherein the pressure source is compressed air.

14. The method of claim 12, wherein the pressure source is hydraulic in nature.

15. The method of claim 8, wherein the lubricant coats the interior of the introducer, the extrusion force applicator, and the implant to reduce friction between the implant, introducer, and extrusion force applicator.

16. A sterilizable volume restricted container for assisting in implanting an implantable device in a body pocket, comprising:
a volume restricted container having a holding portion disposed at distal end of the introducer and a nozzle portion disposed at a proximal end of the introducer body;
an expandable volume reducer mounted over a distal opening of the introducer;
a cap mounted over the expandable volume reducer and configured to be attached to distal end of the volume restricted container;
a port mounted on a distal end of the cap, the port configured to receive a source of gas or fluid;
an injection port disposed at a proximal end of the nozzle port, the injection port configured to provide access to an interior of the introducer body.

17. The sterilizable volume restricted container of claim 16, wherein the sterilizable volume restricted container is reusable.

18. The sterilizable volume restricted container of claim 16, wherein the cap is formed of malleable high heat conductive metal, the malleable high heat conductive metal cap providing increased thermal conduction to contents of the sterilizable volume restricted container and an interior of the sterilizable volume restricted container to reduce dry heat sterilization cycle time.

19. The sterilizable volume restricted container of claim 16, wherein the expandable volume reducer is a diaphragm.

20. The sterilizable volume restricted container of claim 16, wherein the expandable volume reducer is a balloon.

21. The sterilizable volume restricted container of claim 16, wherein the expandable volume reducer is a bellows.

22. The sterilizable volume restricted container of claim 16, wherein the implant contained in the volume restricted container is made with high strength gel with a compressive rupture force greater than 15 kg using a 15 mm diameter probe.

23. The sterilizable volume restricted container of claim 16, wherein the expandable volume reducer is configured to maximize extrusion force and minimize radial compression forces.

24. The sterilizable volume restricted container of claim 16, wherein the expandable volume reducer is a ribbed diaphragm with variations in thickness to direct forces during compression.

25. The sterilizable volume restricted container of claim 16, wherein the expandable volume reducer is a bellows with variations in thickness to direct forces during compression.

26. The sterilizable volume restricted container of claim 16, wherein the expandable volume reducer is a multi-balloon expander configured to direct forces during compression.

27. The sterilizable volume restricted container of claim 16, wherein the expandable volume reducer is a diaphragm wherein the center of the diaphragm is tethered to maximize compressive forces at the edges of the volume restricted container and minimize radial forces during implantation of an implant through the nozzle portion.
